(19) United States
(12) Patent Application Publication
(10) Pub. No.: US 2012/0213769 A1
(43) Pub. Date: Aug. 23, 2012

(54) METHODS OF DIAGNOSING AMYOTROPHIC LATERAL SCLEROSIS (ALS)

(75) Inventors: Miguel Enrique WEIL, Tel-Aviv (IL); Gideon Rechavi, Tel-Aviv (IL)

(73) Assignee: Ramot at Tel-Aviv University Ltd., Tel-Aviv (IL)

(21) Appl. No.: 13/414,904

(22) Filed: Mar. 8, 2012

Related U.S. Application Data
(63) Continuation-in-part of application No. PCT/IL2010/000732, filed on Sep. 6, 2010.
(60) Provisional application No. 61/272,287, filed on Sep. 8, 2009.

(51) Int. Cl. C12Q 1/68; A61P 25/00; A61K 38/02; A61K 31/7088; G01N 33/566; A61K 39/395

(52) U.S. Cl. 424/130.1; 435/6.11; 435/7.24; 435/7.21; 514/17.7; 514/44 R

(57) ABSTRACT

A method of diagnosing amyotrophic lateral sclerosis (ALS) in a subject in need thereof is provided. The method comprising determining in cells of the subject at least one alteration in a sequence or expression level of Cytoplasmic FMR Interacting Protein (CyFIP2; Accession AF160973) and/or Retinoblastoma Binding Protein 9 (RbBP9; Accession AF039564), wherein an altered sequence or expression of the sequence or expression level of the CyFIP2; Accession AF160973) or Retinoblastoma Binding Protein 9 (RbBP9; Accession AF039564) as compared to a control reference sample from a non-ALS subject, is indicative of ALS. The present teachings may be implemented in screening for novel anti-ALS medicaments and for treating ALS.

### RELATED APPLICATION/S

This application is a Continuation-In-Part of PCT Patent Application No. PCT/IL2010/000732 filed Sep. 6, 2010 which claims the benefit of priority under 35 USC 119(e) of U.S. Provisional Patent Application No. 61/272,287 filed Sep. 8, 2009.

### FIELD AND BACKGROUND OF THE INVENTION

The present invention, in some embodiments thereof, relates to methods of diagnosing amyotrophic lateral sclerosis (ALS).

Neurodegenerative diseases represent some of today's most devastating diseases afflicting man. With an aging population, thanks to improved medicine and healthcare, the incidence of diseases such as Alzheimer's disease, multiple sclerosis, Parkinson's disease and other neurodegenerative disorders like Amyotrophic Lateral Sclerosis (ALS) is increasing, and is taking a significant toll on patients, their families and caregivers, healthcare providers and worldwide economies. Besides, most cases of each one of these neurodegenerative disorders are multifactorial, thus the lack of compatible research animal models makes it difficult to study the etiology and molecular mechanisms behind the disease. Identification of a specific disease-related biomarker can help solve this problem and focus efforts toward discovery of effective therapy.

ALS is a fatal and incurable neurodegenerative disorder arising from a progressive loss of motor neurons in the spinal cord, brainstem and motor cortex, resulting in generalized weakness and muscle atrophy. ALS is the most common motor neuron disorder, with a prevalence of approximately 6 per 100,000 at any given time. Approximately 90% of cases are sporadic, the remaining 10% being familial. Over 100 distinct mutations in the ubiquitously expressed enzyme Cu/Zn superoxide dismutase (SOD) have been identified in approximately 20% of familial cases of ALS. The striking pathological and clinical similarity between familial and sporadic disease has sparked enthusiasm that the animal models based on mutant SOD1 might provide insight into mechanisms of both sporadic and familial disease. However, to date, there is no direct evidence validating this assumption. The causes for most cases of ALS are unknown and the clinical course is highly variable, suggesting that multiple factors underlie the disease mechanism (Bruijn et al., 2004, Annu. Rev. Neurosci. 27: 723-49). The complex genetics and unknown etiology of ALS emphasize the need for a multidisciplinary approach to characterize and isolate specific biomarkers found in biological samples from ALS patients.

Mesenchymal stem cells can differentiate into cells of the skeletal system, connective tissue, nervous system, and other tissue. Thus, recently, administration of mesenchymal stem cells has been suggested as treatment for a variety of degenerative diseases, including neurodegenerative disease such as ALS. However, diagnostics based on characteristics of mesenchymal stem cells, and cells of other, non-nervous tissue origin such as white blood cells has not been proposed.

RNA editing is a post-transcriptional mechanism for expanding the proteomic repertoire. A-I RNA editing by enzymes named ADARs (adenosine deaminases acting on RNAs) is essential for normal life and development of both invertebrates and vertebrates. When functioning improperly, this essential process, can lead to pathological conditions ranging from epilepsy to malignant gliomas. So far, only abnormal RNA editing at the Q/R (glutamine/arginine) site of the GluR-2 gene of the AMPA (-amino-3-hydroxy-5-methyl-4-isoxazolepropionate) receptors has been identified to be implicated in ALS (Kawahara et al. 2005 Amyotroph Lateral Scler Other Motor Neuron Disord 6:131-44; Kawahara et al. 2003. Human spinal motoneurons express low relative abundance of GluR2 mRNA: an implication for excitotoxicity in ALS. J Neurochem 85:680-9; Kwak et al. 2005. J Mol Med 83:110-20). It is not yet known if the RNA editing deficiency in the GluR-2 is part of a more global RNA problem related with ADARs activity in ALS.

### SUMMARY OF THE INVENTION

According to an aspect of some embodiments of the present invention there is provided a method of diagnosing amyotrophic lateral sclerosis (ALS) in a subject in need thereof, the method comprising determining in cells of the subject at least one alteration in a sequence or expression level of Cytoplasmic FMR Interacting Protein (CyFIP2; Accession AF160973) and/or Retinoblastoma Binding Protein 9 (RbBP9; Accession AF039564), wherein an altered sequence or expression of the sequence or expression level of the CyFIP2; Accession AF160973) or Retinoblastoma Binding Protein 9 (RbBP9; Accession AF039564) as compared to a control reference sample from a non-ALS subject, is indicative of ALS.

According to some embodiments of the invention, the cells comprise mesenchymal stem cells.

According to some embodiments of the invention, the cells comprise peripheral white blood cells.

According to some embodiments of the invention, the alteration in a sequence or expression level comprises an alteration in RNA editing.

According to some embodiments of the invention, the determining the alteration is effected by an assay selected from the group consisting of PCR, RNA editing assay and PAGE analysis.

According to some embodiments of the invention, the RNA editing assay comprises direct sequencing.

According to some embodiments of the invention, the RNA editing assay assays an A>I editing.

According to some embodiments of the invention, the alteration in expression level comprises increased expression level.

According to some embodiments of the invention, the alteration in expression level comprises decreased expression level.

According to some embodiments of the invention, the determining the expression level is effected at the mRNA level.

According to some embodiments of the invention, the method further comprising substantiating the diagnosis using a diagnosis method selected from the group consisting of electromyography, nerve conduction velocity magnetic resonance imaging (MRI) and bio-molecular analysis.

According to some embodiments of the invention, the method further comprising planning a treatment regimen for the ALS following the step (a).

According to some embodiments of the invention, the method further comprising (b) informing the subject of ALS diagnosis.

According to an aspect of some embodiments of the present invention there is provided a kit for diagnosing ALS comprising means container including at least a pair of oligonucleotide primers capable of specifically binding to any one of CyFIP2 and RbBP9 mRNA sequences or the complement thereof.

According to an aspect of some embodiments of the present invention there is provided a method of screening for a medicament for ALS, the method comprising:

(a) treating cells of an ALS patient with a putative ALS medicament, so as to obtain treated cells; and

(b) determining in the treated cells at least one alteration in a sequence or expression level of Cytoplasmic FMR Interacting Protein (CyFIP2; Accession AF160973) and/or Retinoblastoma Binding Protein 9 (RbBP9; Accession AF039564), wherein an altered sequence or expression of the sequence or expression level of the CyFIP2; Accession AF160973) or Retinoblastoma Binding Protein 9 (RbBP9; Accession AF039564) as compared to a control reference of non-treated cells of the ALS patient, wherein the alteration is indicative of a medicament for ALS.

According to some embodiments of the invention, the cells comprise mesenchymal stem cells.

According to some embodiments of the invention, the alteration comprises up-regulation of the expression and RNA editing.

According to some embodiments of the invention, the cells comprise peripheral blood cells.

According to some embodiments of the invention, the alteration comprises down-regulation of the expression and up-regulation of RNA editing.

According to an aspect of some embodiments of the present invention there is provided a method of treating ALS in a subject in need thereof, the method comprising:

(a) obtaining cells of the subject;

(b) treating the cells with an ALS medicament, so as to obtain treated cells;

(c) determining in the treated cells at least one alteration in a sequence or expression level of Cytoplasmic FMR Interacting Protein (CyFIP2; Accession AF160973) and/or Retinoblastoma Binding Protein 9 (RbBP9; Accession AF039564), wherein an altered sequence or expression of the sequence or expression level of the CyFIP2; Accession AF160973) or Retinoblastoma Binding Protein 9 (RbBP9; Accession AF039564) as compared to a control reference of non-treated cells of the ALS patient, wherein the alteration is indicative of an efficacious medicament for ALS; and

(d) treating the subject with the efficacious medicament.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. [IF IMAGES, REPHRASE] With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-C show analysis of hMSC biological characteristics from ALS patients. FIG. 1A—Comparative analysis of cell survival of hMSC of ALS patients (ALS-hMSC) (n=4) and unaffected normal donors (N-hMSC) (n=2) at two low densities cultured for up to 3 days in the presence or absence of serum. For comparative analysis between different samples and experiments the mean values are expressed as Log of the live/dead ratio as indicated in y axis. The conditions were scored in triplicates of two independent experiments. FIG. 1B—ALS-hMSC and N-hMSC differentiate into adipocytes and osteoblasts. Cells were grown either in adipogenesis or osteogenesis differentiation medium or in control medium (DMEM+FCS) for 21 days. The cells were fixed and stained specifically with oil red for adipocytes or with Alizarin red for osteoblasts to detect differentiated cells. Note that control cells show typical hMSC morphologies and did not stain with the differentiation specific dyes. FIG. 1C—FACS analysis of hMSC surface markers: CD105+, CD90+, CD73+, CD45−, CD19−, CD34−.

FIGS. 2A-B shows mRNA editing levels of CyFIP2 and RbBP9 in ALS-hMSC and N-hMSC. hMSC of 6 ALS patients and 3 of non-ALS donors at passages 3-4 were grown in 10% FBS or in DMEM alone for 5 days (A=ALS, N=normal) (FIG. 2A). RNA was extracted from these cells and cDNA was produced by in vitro reverse transcription for the RNA editing analysis. Editing efficiency of these genes was measured in ALS and non-ALS hMSC and expressed as % of total editing expressed as mean+/− standard error. The level of editing, assessed by direct sequencing, was quantified by the Discovery Studio Gene (DSGene) 1.5 program (Accelrys Inc.). The reliability of this method was further verified by cloning of individual sequences. PCR products were ligated into pGem-T Easy (Promega). After transformation of Escherichia coli, DNA was extracted using QIAprep Spin (QIAGEN), and individual plasmids were sequenced. The percentage of the edited clones was determined and compared to the DSGene quantification. This approach confirmed that the quantification was reliable even at low editing ratios of 5%, representing the transcript repertoire and not background noise. Statistical analysis of the significant difference between ALS and non-ALS groups for the three genes under the two different culture conditions was performed by T-test after square root transformation. In DMEM the significance for the two genes was: CyFIP2 p=0.01; RbBP9 p=0.001. In FBS the significance was only for CyFIP2 p=0.019 FIG. 2B shows RT-PCR results of the expression of CyFIP2 and RbBP9 in RNA extracts of 6 ALS-hMSC and 6 N-hMSC to confirm the expression of both genes in the samples that were analyzes in FIG. 2A with the addition of three new N-hMSC samples. GAPDH (serves as positive control). No cDNA: PCR reaction without template. All the transcripts were detected using specific primers and cDNA was synthesized by reverse transcription from RNA extracted from hMSC cultured in 10% FBS or in DMEM alone. The specificity of the PCR products was confirmed by DNA sequencing.

FIG. 3 shows quantitative RT-PCR analysis of the expression of CyFIP2 and RbBP9 in RNA extracts of 6 ALShMSC and 6 N-hMSC. Boxplot diagram shows the quantitative RT-PCR (QPCR) analysis of hMSC cultured in DMEM+10% FBS. RLF10 served as normalizing gene for these cells. All the transcripts were detected using specific primers adequate for QPCR and cDNA was synthesized from RNA as described in FIGS. 2A-B. The results are presented as mean ±sd of relative expression of mRNA levels in arbitrary units (Log 2X). Ttest with two sample equal variance after square root transformation was performed to establish statistical significance between the groups ALS and Normal. p=0.0028 for CyFIP2; p=0.0021 for RbBP9.

FIG. 4 shows quantitative RT-PCR analysis of the expression of CyFIP2 and RbBP9 in RNA extracts of PBL from ALS patients (ALS-PBL) and from non-ALS donors (N-PBL). Boxplot diagram shows the QPCR results,RS9 served as normalizing gene for these cells. All the transcripts were detected using specific primers adequate for QPCR and cDNA was synthesized by reverse transcription from RNA extracted from PBL fraction isolated from peripheral blood samples from 17 ALS and 19 healthy individuals. The results are presented as mean ±sd of relative expression of mRNA levels in arbitrary units (Log2X). T-test with two sample equal variance after square root transformation was performed to establish statistical significance between the groups ALS and Normal. p=0.000062 for CyFIP2; p=0.042 for RbBP9.

FIGS. 5A-B show Western blot analysis of the expression of CyFIP2 and RbBP9 in protein lysates of ALS non-ALS hMSC. ALS hMSC from 4 ALS patients and non-ALS hMSC from 5 control individuals at the same passage number were cultured in DMEM 10% FBS until they reached 80% confluence. Cell lysate was extract and separated by SDS-PAGE, transferred to nitrocellulose membranes for Western blot analysis with either rabbit anti CyFIP2 or rabbit anti RbBP9 antibodies. β-actin was used as loading control for the experiments. FIG. 5A shows the ECL autoradiograms of a representative WB experiment. FIG. 5B shows the densitometry analysis of the WB results for CyFIP2 and RbBP9 respectively. The results are represented as mean values±s.d. Statistical analysis of the results was performed using T-test with two sample equal variance. Asterisks represent significant differences between protein levels in ALS and non-ALS cells. p<0.05 for RbBP9; p<0.001 for CyFIP2.

### DESCRIPTION OF SPECIFIC EMBODIMENTS OF THE INVENTION

The present invention, in some embodiments thereof, relates to methods of diagnosing amyotrophic lateral sclerosis (ALS)

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Amyotrophic lateral sclerosis (ALS) is a progressive, neurodegenerative disorder caused by the degeneration of motor neurons of the spinal cord and brain. The causes of most cases of ALS are unknown and the clinical course is highly variable. This fact enhances the need to isolate specific biomarkers found in biological samples of ALS patients.

The present inventors envisaged that mesenchymal stem cells (MSCs) can be used as a robust source for the isolation of such markers. The present inventors found that although MSCs of ALS patients are seemingly similar to those of normal subjects as evidenced by survival and differentiation potential, they manifest different expression levels and RNA editing levels of specific genes i.e., CyFIP2 and RbBP9. These genes can therefore be used as putative markers for ALS.

While further reducing the present invention to practice, the present inventors found that these markers are also differentially expressed on peripheral blood lymphocytes and as such can be readily used as accessible biomarkers for ALS.

Thus, according to an aspect of the invention there is provided a method of diagnosing amyotrophic lateral sclerosis (ALS) in a subject in need thereof, the method comprising determining in cells of the subject at least one alteration in a sequence or expression level of Cytoplasmic FMR Interacting Protein (CyFIP2; Accession AF160973) and/or Retinoblastoma Binding Protein 9 (RbBP9; Accession AF039564), wherein an altered sequence or expression of said sequence or expression level of said CyFIP2; Accession AF160973) or Retinoblastoma Binding Protein 9 (RbBP9; Accession AF039564) as compared to a control reference sample from a non-ALS subject, is indicative of ALS.

As used herein “amyotrophic lateral sclerosis (ALS)” also referred to as “Lou Gehrig's disease” refers to a progressive, fatal, neurodegenerative disease caused by the degeneration of motor neurons, the nerve cells in the central nervous system that control voluntary muscle movement. The disorder causes muscle weakness and atrophy throughout the body as both the upper and lower motor neurons degenerate, ceasing to send messages to muscles. Unable to function, the muscles gradually weaken, develop fasciculations (twitches) because of denervation, and eventually atrophy because of that denervation. Affected subjects may ultimately lose the ability to initiate and control all voluntary movement; bladder and bowel sphincters and the muscles responsible for eye movement are usually, but not always, spared.

Cognitive function is generally spared except in certain situations such as when ALS is associated with frontotemporal dementia. However, there are reports of more subtle cognitive changes of the frontotemporal type in many patients when detailed neuropsychological testing is employed. Sensory nerves and the autonomic nervous system, which controls functions like sweating, generally remain functional. ALS as used herein refers to all the above exemplary manifestations. ALS, as used herein refers to hereditary and sporadic ALS.

As used herein “a subject in need thereof” refers to a human subject who is at risk of developing ALS or who exhibits clinical signs of ALS. The subject is of any age and gender.

Following is an exemplary description of hereditary causes as well as environmental risks who may contribute to onset of disease.

An inherited genetic defect on chromosome 21 (coding for superoxide dismutase) is associated with approximately 20% of familial cases of ALS. This mutation is believed to be autosomal dominant. The most common ALS causing SOD1 mutation in North America is A4V, characterized by an exceptionally rapid progression from onset to death. The children of those diagnosed with familial ALS have a higher risk factor for developing the disease; however, those who have close family members diagnosed with sporadic ALS have no greater a risk factor than the general population, suggesting an environmental or other non-genetic cause.

Some environmental causative factors have been suggested for the increased incidence of ALS. These include, prolonged exposure to a dietary neurotoxin called BMAA produced by cyanobacteria which is one of several possible neurotoxic compounds found in the seed of the cycad Cycas circinalis, a tropical plant found in Guam; Exposure to pesticides; toxic exposure such as nerve gas.

As used herein the term “diagnosing” refers to determining presence or absence of a pathology (i.e., ALS), classifying a pathology or a symptom, determining a severity of the pathology, monitoring pathology progression, forecasting an outcome of a pathology and/or prospects of recovery and screening of a subject for a specific disease.

According to some embodiments of the invention, diagnosing refers to population screening i.e., screening subjects for ALS which is followed by substantiation of the screen results using gold standard methods, as further described hereinbelow.

As used herein “Cytoplasmic FMR Interacting Protein (CyFIP2; Accession AF160973, NM_{—}001037332)” refers to SEQ ID NOs: 33 and 34.

As used herein “Retinoblastoma Binding Protein 9 (RbBP9; Accession AF039564, NM_{—}006606)” refers to SEQ ID NO: 35 and 36.

As used herein, the term “alteration in a sequence” refers to an alteration in the mRNA level typically as a result of RNA editing. In this case the alteration in sequence is a result of reduced adenosine (A) to inosine (I) RNA editing. The change is monitored statistically. The change in the sequence can be assayed at the RNA level or protein level, if there is a manifestation in the latter.

As used herein “alteration in expression level” refers to a change in expression at the DNA, mRNA and optionally protein level. The alteration can be increased or decreaed expression as compared to a control reference cell (of the same type) from a subject who is not affected with ALS. Increased or decreased level of expression can result from for example, but not limited in anyway to, gene amplification, nonsense, missense, frameshift mutations, enhanced or deficient transcription/translation, enhanced or deficient turnover of RNA or protein or alternate RNA editing.

As mentioned alteration in a sequence or expression level is determined in cells of the subject. The assay can be done in vivo, ex-vivo (using cells retieved from the subject), or in vitro (such as using cells from animal models).

According to a specific embodiment the cells comprise mesenchymal stem cells.

Mesenchymal stem cells are the formative pluripotent blast cells. Mesenchymal stem cells (MSCs) give rise to one or more mesenchymal tissues (e.g., adipose, osseous, cartilaginous, elastic and fibrous connective tissues, myoblasts, cardiac like cells) as well as to tissues other than those originating in the embryonic mesoderm (e.g., neural cells) depending upon various influences from bioactive factors such as cytokines. MSCs can be isolated from embryonic yolk sac, placenta, umbilical cord, fetal and adolescent skin, blood, bone marrow, adipose and other tissues, although their abundance in the bone marrow far exceeds their abundance in other tissues. MSCs have been shown to have immunosuppressive functions in various settings, including autoimmune diseases and transplantation, rendering liposomes generated therefrom ultimate tools in inflammatory and autoimmune settings.

Methods of isolating, purifying and expanding mesenchymal stem cells (MSCs) are known in the arts and include, for example, those disclosed by Caplan and Haynesworth in U.S. Pat. No. 5,486,359 and Jones E. A. et al., 2002, Isolation and characterization of bone marrow multipotential mesenchymal progenitor cells, Arthritis Rheum. 46(12): 3349-60.

According to another specific embodiment, the cells comprise peripheral blood lymphocytes.

As used herein, the phrase “peripheral blood lymphocytes” refers to a sample taken from circulating blood as opposed to blood cells sequestered within the lymphatic system, spleen, liver, or bone marrow. The term refers to large granular lymphocytes and small lymphocytes. Large granular lymphocytes include natural killer cells (NK cells). Small lymphocytes consist of T cells and B cells.

As used herein the term “isolated” refers to isolated from the natural environment. According to a specific embodiment, the term relates to serum purified i.e., no plasma.

Peripheral blood cell samples are typically taken using a syringe with a needle.

Methods of processing peripheral blood cell samples are known in the art and further described in the Examples section herein below.

According to a specific embodiment, when the cells comprise mesenchymal stem cells, the alteration in expression level comprises decreased expression level.

According to a specific embodiment, when the cells comprise PBLs, the alteration in expression level comprises increased expression level.

Regardless of the cells, methods of determining levels of gene expression are well known in the art, some are summarized infra. Specific primer sequences (served as mere examples) are provided in the Examples section which follows.

Methods of Detecting the Expression Level of RNA

The expression level of the RNA in the cells of the present invention can be determined using methods known in the arts.

Northern Blot analysis: This method involves the detection of a particular RNA in a mixture of RNAs. An RNA sample is denatured by treatment with an agent (e.g., formaldehyde) that prevents hydrogen bonding between base pairs, ensuring that all the

RNA molecules have an unfolded, linear conformation. The individual RNA molecules are then separated according to size by gel electrophoresis and transferred to a nitrocellulose or a nylon-based membrane to which the denatured RNAs adhere. The membrane is then exposed to labeled DNA probes. Probes may be labeled using radio-isotopes or enzyme linked nucleotides. Detection may be using autoradiography, colorimetric reaction or chemiluminescence. This method allows both quantitation of an amount of particular RNA molecules and determination of its identity by a relative position on the membrane which is indicative of a migration distance in the gel during electrophoresis.

RT-PCR analysis: This method uses PCR amplification of relatively rare RNAs molecules. First, RNA molecules are purified from the cells and converted into complementary DNA (cDNA) using a reverse transcriptase enzyme (such as an MMLV-RT) and primers such as, oligo dT, random hexamers or gene specific primers. Then by applying gene specific primers and Taq DNA polymerase, a PCR amplification reaction is carried out in a PCR machine. Those of skills in the art are capable of selecting the length and sequence of the gene specific primers and the PCR conditions (i.e., annealing temperatures, number of cycles and the like) which are suitable for detecting specific RNA molecules. It will be appreciated that a semi-quantitative RT-PCR reaction can be employed by adjusting the number of PCR cycles and comparing the amplification product to known controls.

Real-time PCR—real-time polymerase chain reaction, also called quantitative real time polymerase chain reaction (Q-PCR/qPCR/qrt-PCR) or kinetic polymerase chain reaction (KPCR), is a laboratory technique based on the PCR, which is used to amplify and simultaneously quantify a targeted DNA molecule. It enables both detection and quantification (as absolute number of copies or relative amount when normalized to DNA input or additional normalizing genes) of one or more specific sequences in a DNA sample.

The procedure follows the general principle of polymerase chain reaction; its key feature is that the amplified DNA is detected as the reaction progresses in real time, a different approach compared to standard PCR, where the product of the reaction is detected at its end. Two common methods for detection of products in real-time PCR are: (1) non-specific fluorescent dyes that intercalate with any double-stranded DNA, and (2) sequence-specific DNA probes consisting of oligonucleotides that are labeled with a fluorescent reporter which permits detection only after hybridization of the probe with its complementary DNA target. Frequently, real-time PCR is combined with reverse transcription to quantify messenger RNA and Non-coding RNA in cells or tissues.

Abbreviations used for real-time PCR methods vary widely and include RTQ-PCR, Q-PCR or qPCR. Real-time reverse-transcription PCR is often denoted as qRT-PCR, RRT-PCR, or RT-rt PCR.

RNA in situ hybridization stain: In this method DNA or RNA probes are attached to the RNA molecules present in the cells. Generally, the cells are first fixed to microscopic slides to preserve the cellular structure and to prevent the RNA molecules from being degraded and then are subjected to hybridization buffer containing the labeled probe. The hybridization buffer includes reagents such as formamide and salts (e.g., sodium chloride and sodium citrate) which enable specific hybridization of the DNA or RNA probes with their target mRNA molecules in situ while avoiding non-specific binding of probe. Those of skills in the art are capable of adjusting the hybridization conditions (i.e., temperature, concentration of salts and formamide and the like) to specific probes and types of cells. Following hybridization, any unbound probe is washed off and the slide is subjected to either a photographic emulsion which reveals signals generated using radio-labeled probes or to a colorimetric reaction which reveals signals generated using enzyme-linked labeled probes.

In situ RT-PCR stain: This method is described in Nuovo G J, et al. [Intracellular localization of polymerase chain reaction (PCR)-amplified hepatitis C cDNA. Am J Surg Pathol. 1993, 17: 683-90] and Komminoth P, et al. [Evaluation of methods for hepatitis C virus detection in archival liver biopsies. Comparison of histology, immunohistochemistry, in situ hybridization, reverse transcriptase polymerase chain reaction (RT-PCR) and in situ RT-PCR. Pathol Res Pract. 1994, 190: 1017-25]. Briefly, the RT-PCR reaction is performed on fixed cells by incorporating labeled nucleotides to the PCR reaction. The reaction is carried on using a specific in situ RT-PCR apparatus such as the laser-capture microdissection PixCell I LCM system available from Arcturus Engineering (Mountainview, Calif.).

Oligonucleotide microarray—In this method oligonucleotide probes capable of specifically hybridizing with the polynucleotides of the present invention are attached to a solid surface (e.g., a glass wafer). Each oligonucleotide probe is of approximately 20-25 nucleic acids in length. To detect the expression pattern of the polynucleotides of the present invention in a specific cell sample (e.g., blood cells), RNA is extracted from the cell sample using methods known in the art (using e.g., a TRIZOL solution, Gibco BRL, USA). Hybridization can take place using either labeled oligonucleotide probes (e.g., 5′-biotinylated probes) or labeled fragments of complementary DNA (cDNA) or RNA (cRNA). Briefly, double stranded cDNA is prepared from the RNA using reverse transcriptase (RT) (e.g., Superscript II RT), DNA ligase and DNA polymerase I, all according to manufacturer's instructions (Invitrogen Life Technologies, Frederick, Md., USA). To prepare labeled cRNA, the double stranded cDNA is subjected to an in vitro transcription reaction in the presence of biotinylated nucleotides using e.g., the BioArray High Yield RNA Transcript Labeling Kit (Enzo, Diagnostics, Affymetix Santa Clara Calif.). For efficient hybridization the labeled cRNA can be fragmented by incubating the RNA in 40 mM Tris Acetate (pH 8.1), 100 mM potassium acetate and 30 mM magnesium acetate for 35 minutes at 94° C. Following hybridization, the microarray is washed and the hybridization signal is scanned using a confocal laser fluorescence scanner which measures fluorescence intensity emitted by the labeled cRNA bound to the probe arrays.

For example, in the Affymetrix microarray (Affymetrix®, Santa Clara, Calif.) each gene on the array is represented by a series of different oligonucleotide probes, of which, each probe pair consists of a perfect match oligonucleotide and a mismatch oligonucleotide. While the perfect match probe has a sequence exactly complimentary to the particular gene, thus enabling the measurement of the level of expression of the particular gene, the mismatch probe differs from the perfect match probe by a single base substitution at the center base position. The hybridization signal is scanned using the Agilent scanner, and the Microarray Suite software subtracts the non-specific signal resulting from the mismatch probe from the signal resulting from the perfect match probe.

Methods of Detecting Gene Amplification Level

Southern blot analysis: is a method routinely used in molecular biology for detection of a specific DNA sequence in DNA samples. Southern blotting combines transfer of electrophoresis-separated DNA fragments to a filter membrane and subsequent fragment detection by probe hybridization. Southern blots performed with restriction enzyme-digested genomic DNA may be used to determine the number of sequences (e.g., gene copies) in a genome. A probe that hybridizes only to a single DNA segment that has not been cut by the restriction enzyme will produce a single band on a Southern blot, whereas multiple bands will likely be observed when the probe hybridizes to several highly similar sequences (e.g., those that may be the result of sequence duplication). Modification of the hybridization conditions (for example, increasing the hybridization temperature or decreasing salt concentration) may be used to increase specificity and decrease hybridization of the probe to sequences that are less than 100% similar (see also Southern, Edwin Mellor (5 Nov. 1975). “Detection of specific sequences among DNA fragments separated by gel electrophoresis”. Journal of Molecular Biology 98 (3): 503-517, which is hereby incorporated by reference in its entirety).

DNA Microarrays/DNA Chips:

The expression of thousands of genes may be analyzed simultaneously using DNA microarrays, allowing analysis of the complete transcriptional program of an organism during specific developmental processes or physiological responses. DNA microarrays consist of thousands of individual gene sequences attached to closely packed areas on the surface of a support such as a glass microscope slide. Various methods have been developed for preparing DNA microarrays. In one method, an approximately 1 kilobase segment of the coding region of each gene for analysis is individually PCR amplified. A robotic apparatus is employed to apply each amplified DNA sample to closely spaced zones on the surface of a glass microscope slide, which is subsequently processed by thermal and chemical treatment to bind the DNA sequences to the surface of the support and denature them. Typically, such arrays are about 2×2 cm and contain about individual nucleic acids 6000 spots. In a variant of the technique, multiple DNA oligonucleotides, usually 20 nucleotides in length, are synthesized from an initial nucleotide that is covalently bound to the surface of a support, such that tens of thousands of identical oligonucleotides are synthesized in a small square zone on the surface of the support. Multiple oligonucleotide sequences from a single gene are synthesized in neighboring regions of the slide for analysis of expression of that gene. Hence, thousands of genes can be represented on one glass slide. Such arrays of synthetic oligonucleotides may be referred to in the art as “DNA chips”, as opposed to “DNA microarrays”, as described above [Lodish et al. (eds.). Chapter 7.8: DNA Microarrays: Analyzing Genome-Wide Expression. In: Molecular Cell Biology, 4th ed., W. H. Freeman, New York. (2000)].

Methods of Detecting Expression and/or Activity of Proteins

Expression and/or activity level of proteins expressed in the cells of the cultures of the present invention can be determined using methods known in the arts.

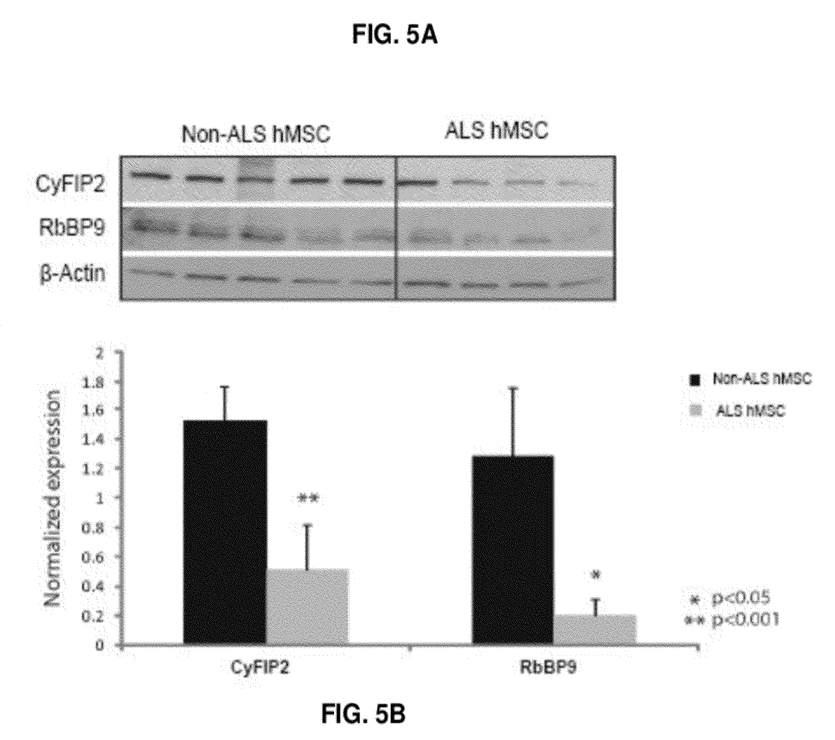

Antibodies to RbBP9 and CyFIP2 are available from commercial sources. For example from Millipore, Sigma-Aldrich, R&D Systems, Cell Signaling Technology, Abnova, OriGene, Novus Biologicals, and/or Epitomics). Proteintech group Inc. CYFIP2 Antibody catalog number: 18011-1-AP and RbBP9 Antibody catalog number: 12230-2-AP (see e.g. the example section which follows and FIGS. 5A-B).

Enzyme linked immunosorbent assay (ELISA): This method involves fixation of a sample (e.g., fixed cells or a proteinaceous solution) containing a protein substrate to a surface such as a well of a microtiter plate. A substrate specific antibody coupled to an enzyme is applied and allowed to bind to the substrate. Presence of the antibody is then detected and quantitated by a colorimetric reaction employing the enzyme coupled to the antibody. Enzymes commonly employed in this method include horseradish peroxidase and alkaline phosphatase. If well calibrated and within the linear range of response, the amount of substrate present in the sample is proportional to the amount of color produced. A substrate standard is generally employed to improve quantitative accuracy.

Western blot: This method involves separation of a substrate from other protein by means of an acrylamide gel followed by transfer of the substrate to a membrane (e.g., nylon or PVDF, see e.g. ‘materials and methods’ section hereinbelow). Presence of the substrate is then detected by antibodies specific to the substrate, which are in turn detected by antibody binding reagents. Antibody binding reagents may be, for example, protein A, or other antibodies. Antibody binding reagents may be radiolabeled or enzyme linked as described hereinabove. Detection may be by autoradiography, colorimetric reaction or chemiluminescence. This method allows both quantitation of an amount of substrate and determination of its identity by a relative position on the membrane which is indicative of a migration distance in the acrylamide gel during electrophoresis.

Radio-immunoassay (RIA): In one version, this method involves precipitation of the desired protein (i.e., the substrate) with a specific antibody and radiolabeled antibody binding protein (e.g., protein A labeled with I¹²⁵) immobilized on a precipitable carrier such as agarose beads. The number of counts in the precipitated pellet is proportional to the amount of substrate.

In an alternate version of the RIA, a labeled substrate and an unlabelled antibody binding protein are employed. A sample containing an unknown amount of substrate is added in varying amounts. The decrease in precipitated counts from the labeled substrate is proportional to the amount of substrate in the added sample.

Fluorescence activated cell sorting (FACS): This method involves detection of a substrate in situ in cells by substrate specific antibodies. The substrate specific antibodies are linked to fluorophores. Detection is by means of a cell sorting machine which reads the wavelength of light emitted from each cell as it passes through a light beam. This method may employ two or more antibodies simultaneously.

Immunohistochemical analysis: This method involves detection of a substrate in situ in fixed cells by substrate specific antibodies. The substrate specific antibodies may be enzyme linked or linked to fluorophores. Detection is by microscopy and subjective or automatic evaluation. If enzyme linked antibodies are employed, a colorimetric reaction may be required. It will be appreciated that immunohistochemistry is often followed by counterstaining of the cell nuclei using for example Hematoxyline or Giemsa stain.

RNA Editing Assay

The level of editing can be assessed by direct sequencing and quantified by the Discovery Studio Gene (DSGene) 1.5 program (ASccelrys).

Alternatively or additionally, RNA editing level can be determined by cloning of specific sequences. PCR products are ligated into bacterial expression vectors. After bacterial transformation, DNA is extracted and individual plasmids sequenced. The percentage of edited clones is determined and according to some embodiments compared to DSGene quantification.

Another exemplary assay of RNA editing is provided by Roberson and Rosenthal RNA 2006. 12: 1907-1912, which Is hereby incorporated by reference in its entirety.

Once diagnosis has been made, the results are recorded and the subject is informed of the diagnosis (ALS positive or negative).

Diagnosis may be augmented such as by using Gold-standard or routine methods. Examples include but are not limited to electromyography, nerve conduction velocity magnetic resonance imaging (MRI) and bio-molecular analysis.

Molecular markers used for ALS diagnosis are taught in U.S. Patent Application Number 20100152125 and 20070202537 teaching specific mutations in Superoxide Dismutase (SOD) gene, each of which is hereby incorporated by reference.

Once diagnosis has been established the method of the present invention further contemplates planning a treatment regimen for ALS.

Current treatment modalities for ALS include but are not limited to, FDA approved medications such as Riluzole (Rilutek). Riluzole is believed to reduce damage to motor neurons by decreasing the release of glutamate via activation of glutamate transporters. In addition, the drug offers a wide array of other neuroprotective effects, by means of sodium and calcium channel blockades, inhibition of protein kinase C, and the promotion of NMDA (N-methyl d-aspartate) receptor antagonism. In addition to the use of medical intervention to treat ALS, there are other approaches applied for ALS patients. These include mind body methods, holistic problem solving methods, psychotherapy, hypnotherapy and nutrition related methods.

As noted, the invention also provides a kit for detecting ALS in a patient. In one embodiment, the kit includes at least one container means. The container means contains a system for detecting ALS in a patient, wherein the system comprises at least a pair of oligonucleotide primers capable of specifically binding to any one of CyFIP2 and RbBP9; Accession AF039564. A kit of this invention may include additional components, as needed, including suitable buffers, indicators (e.g., fluorophores, chromophores or enzymes providing same), controls (e.g., a suitable polynucleotide of this invention) and directions for using the kit. Kit components can be provided in nearly any acceptable form, including a liquid or solid, e.g, as a lyophilized powder. In an alternative or additional embodiment the kit may comprise antibodies for detecting alteration in polypeptide (CyFIP2 and RbBP9) expression.

The kit may be formulated as an array.

In one embodiment, the array comprises at least 2 and no more than 100 antibodies for determining a gene expression profile of a biological sample, wherein at least one of said antibodies or antibody fragments is selected capable of binding with a protein product of CyFIP2 and at least one of said antibodies or antibody fragments is selected capable of binding with a protein product of RbBP9.

In another embodiment, the array comprises at least 2 and no more than 100 polynucleotide sequences for determining a gene expression profile of a biological sample, wherein at least one of said sequences is selected capable of hybridizing with a transcription product of a polynucleotide sequence of CyFIP2 and at least one of said sequences is selected capable of hybridizing with a transcription product of a polynucleotide sequence of RbBP9.

The present teachings can be exploited for screening new anti ALS medicaments.

As used herein an “anti ALS medicament ” (interchangeably used with ALS medicament) refers to a medicament which halts the disease, ameliorates symptoms or cure the disease. The (putative) can be a small molecule drug, a biological compostion (e.g., peptide, polypeptide, nucleic acid agent, antibody) or a combination thereof.

Thus, according to an aspect of the invention there is provided a method of screening for a medicament for ALS, the method comprising:

(a) treating cells of an ALS patient with a putative ALS medicament, so as to obtain treated cells; and

(b) determining in the treated cells at least one alteration in a sequence or expression level of Cytoplasmic FMR Interacting Protein (CyFIP2; Accession AF160973) and/or Retinoblastoma Binding Protein 9 (RbBP9; Accession AF039564), wherein an altered sequence or expression of the sequence or expression level of the

CyFIP2; Accession AF160973) or Retinoblastoma Binding Protein 9 (RbBP9; Accession AF039564) as compared to a control reference of non-treated cells of the ALS patient, wherein the alteration is indicative of a medicament for ALS.

The sequence alteration is determined as described above.

In one embodiment, the cells comprise mesenchymal stem cells. In such a case, the alteration comprises up-regulation of the expression and RNA editing.

Alternatively or additionally. the cells comprise peripheral blood cells. Accordingly, the alteration (in PBLs) comprises down-regulation of the expression and up-regulation of RNA editing.

Once a putative drug is established as a medicament for ALS, the present teachings also contemplate its synthesis (and/or purification) and use of same in personalized therapy settings.

Accordingly, the present invention further provides for a method of treating ALS in a subject in need thereof, the method comprising:

(a) obtaining cells of the subject;

(b) treating the cells with an ALS medicament, so as to obtain treated cells;

(c) determining in the treated cells at least one alteration in a sequence or expression level of Cytoplasmic FMR Interacting Protein (CyFIP2; Accession AF160973) and/or Retinoblastoma Binding Protein 9 (RbBP9; Accession AF039564), wherein an altered sequence or expression of the sequence or expression level of the CyFIP2; Accession AF160973) or Retinoblastoma Binding Protein 9 (RbBP9; Accession AF039564) as compared to a control reference of non-treated cells of the ALS patient, wherein the alteration is indicative of an efficacious medicament for ALS; and

(d) treating the subject with the efficacious medicament.

Thus the present teachings relate to new ALS markers and use thereof in diagnosis of ALS, screening for new medicaments and treatment of the disease.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials and Methods

Isolation and Culture of hMSC

Bone marrow samples were obtained from the iliac crest's bone marrow of healthy donors ranging in age from 20-56 years and of six male ALS patients (age 43-56) who had signed for consent according to the guidelines of the ethics committee of the Laniado Hospital supervised by the Israeli Health Ministry Ethics Committee. The aspirates were diluted 1:2 with Hanks' Balance Salt Solution (Biological Industries, Israel). The mononuclear cell layer was isolated from red blood cells by density gradient using UNI-SEP maxi tubes (Novamed, Israel) centrifuged at 1000 g for 20 minutes. The cell pellet was re-suspended in culture Dulbecco's Modified Eagle's Medium (DMEM) (Gibco, Invitrogen, NY, USA) supplemented with 1 mM MEM Sodium Pyruvate (Gibco, Invitrogen, NY, USA), 1% Penicillin-streptomycin-nystatin solution (Biological Industries, Israel), 10% heat inactivated Fetal Bovine Serum (FBS) (Hyclone, USA) and cultured in polystyrene plastic 75 cm² tissue culture flasks (Corning, NY, USA) at 37° C. with 5% CO₂. hMSC were isolated by their characteristic adherence to the plastic while other cells were washed through successive medium replacement as described by Blondheim et al. (Blondheim et al., 2006, Stem Cells Dev. 15: 141-164). Cell passages and expansion of hMSC cultures were made when cells reached 80-100% confluence by applying 0.025% Trypsin-EDTA solution B (Biological Industries, Israel) for few minutes, and then splitting the suspended cells into three new 75 cm² tissue culture flasks (Corning, NY, USA). In this study, hMSC ranging from passages 3 to 6 were used.

Viability Test

Cell viability was assayed as previously reported (Solmesky et al., 2009, BMP signaling is involved in human mesenchymal stem cells survival in serum free medium. Stem Cells and Dev. Vol. 18, pp. 1283-1291). At the end of incubation with the corresponding treatments, the medium was supplemented with 25 μl/well of a suspension made of 20 μg/ml Calcein AM (Invitrogen, Oregon, USA) (a cell permeable non fluorescent dye that undergoes conversion to a green fluorescent calcein by intracellular esterases hydrolysis), 2 μg/ml Bisbenzimide (Hoechst 33342) (Sigma, MO, USA) (which is intended to stain all the nuclei), and 4 μg/ml propidium iodide (PI) (Sigma, MO, USA) (a non cell-permeable dye which is intended to stain only the apoptotic cells) in PBS. The stained cells were incubated for 20 minutes at 37° C. and then the proportion of viable cells was determined by counting calcein positive (viable) cells out of the total Hoechst positive stained nuclei. The difference in the number of cells score between them was confirmed by counting the number of PI positive stained nuclei. All quantifications and images acquisition were made using a Nikon DXM1200f digital camera (Nikon, Japan) mounted on an inverted fluorescence Nikon Eclipse TE2000-S microscope (Nikon, Japan) using a 10× magnification objective.

Cell Differentiation to Osteocytes and Adipocytes

The procedures were based on the protocols established by Pittenger et al. (Pittenger et al., 1999, Science 284: 143-7). For osteocyte differentiation, hMSC were plated at a cell density of 60,000 cells per well, in 24 wells plates and grown to confluence. Then the cells were cultured during 14-17 days, changing the medium every 2-3 days, with a medium composed of Dulbecco's Modified Eagle's Medium (DMEM) (Gibco, Invitrogen, NY, USA) supplemented with 1 mM MEM Sodium Pyruvate (Gibco, Invitrogen, NY, USA), 1% Penicillin-streptomycin-nystatin solution (Biological Industries, Israel), 10% heat inactivated Fetal Bovine Serum (FBS) (Hyclone, USA), 0.1 μM dexamethasone (Sigma, MO, USA), 0.2mM ascorbic acid 2-phosphate (Sigma, MO, USA), and 10mM glicerol 2-phosphate (Sigma, MO, USA). The resulting osteocytes were fixed in 70% ethanol and stained with Alizarin Red solution (Sigma, MO, USA). For adipocyte differentiation, the cells were cultured during 21 days in 24 wells plates at 60,000 cells/well using the following medium change schedule since 100% confluence was reached: days 1, 3, 5: induction medium; day 7: maintenance medium; days 9, 11, 13: induction medium; day 15: maintenance medium; days 17, 19, 21: induction medium. Induction medium was composed of Dulbecco's Modified Eagle's Medium (DMEM) (Gibco, Invitrogen, NY, USA) supplemented with 1 mM MEM Sodium Pyruvate (Gibco, Invitrogen, NY, USA), 1% Penicillin-streptomycin-nystatin solution (Biological Industries, Israel), 10% heat inactivated Fetal Bovine Serum (FBS) (Hyclone, USA), 1 μM dexamethasone (Sigma, MO, USA), 0.5 mM IBMX (Sigma, MO, USA), 10 μg/ml insulin (Sigma, MO, USA), 100 μM indomethacine (Sigma, MO, USA). Maintenance medium was composed of Dulbecco's Modified Eagle's Medium (DMEM) (Gibco, Invitrogen, NY, USA) supplemented with 1 mM MEM Sodium Pyruvate (Gibco, Invitrogen, NY, USA), 1% Penicillin-streptomycin-nystatin solution (Biological Industries, Israel), 10% heat inactivated Fetal Bovine Serum (FBS) (Hyclone, USA), and 10 μg/ml insulin (Sigma, MO, USA). The resulting adipocytes were fixed in 4% paraformaldehyde and stained with Oil Red O solution (Sigma, MO, USA) whereas the nuclei were stained with Hoescht 33342 (Sigma, MO, USA).

RNA Editing

The level of editing, assessed by direct sequencing, was quantified by the Discovery Studio Gene (DSGene) 1.5 program (Accelrys Inc.). The reliability of this method was further verified by cloning of individual sequences. PCR products were ligated into pGem-T Easy (Promega). After transformation of Escherichia coli, DNA was extracted using QIAprep Spin (QIAGEN), and individual plasmids were sequenced. The percentage of the edited clones was determined and compared to the DSGene quantification. This approach confirmed that the quantification was reliable even at low editing ratios of <5%, representing the transcript repertoire and not background noise.

RT-PCR

After removal of the culture media the cells were washed with PBS and trypsinized as described above. RNA was isolated from the cells using EZ-RNA (Biological Industries, Israel) according to the manufacturer's instructions.

The cDNAs were synthesized from at least 600 ng RNA using the Reverse-IT first strand synthesis kit (Abgene, Surrey, UK) and each PCR reaction was performed using the Reddymix PCR master mix (Abgene, Surrey, UK) according to the manufacturer's instructions. The different pairs of specific primers listed below were added at a final concentration of 2 μM in a 30 μl reaction volume mix. The primers for the PCR were designed to bind sequences from different exons to avoid false positives due to genomic DNA contamination. Two rounds of PCR were carried out adding reddymix prior to the second round. In each round, the DNA was denatured at 95° C. for 5 minutes and then subjected to 25 cycles of 1 minute at 95° C., 1 minute at annealing temperature of 56° C., and 1 minute at 72° C. Then, the reaction was subjected for 10 minutes to 72° C. The identity of the PCR products which showed the expected band size was confirmed by DNA sequencing. The primers used in this study were synthesized by Syntezza, Israel and were the following (the annealing temperature and the product size are indicated in parenthesis):

GluR2
(SEQ ID NO: 32)
Forward: 5′-ACCCTTACTGAGCTCCCTTC-3′,
GluR2
(SEQ ID NO: 1)
Reverse: 5′-CTTTCCCATATACAAGTTCTCCA-3′,
(55,7° C., 286 bp);
CYFIP2
(SEQ ID NO: 2)
Forward: 5′-GCTGGTTCATCCCACAGACA-3′,
CYFIP2
(SEQ ID NO: 3)
Reverse: 5′-AGGACAATGGGTCCATCCAG-3′,
(53.6° C., 532 bp);
RbBP9
(SEQ ID NO: 4)
Forward: 5′-GCTGCACTCATGGCTTCTCC-3′,
RbBP9
(SEQ ID NO: 5)
Reverse: 5′-AAAGTGGCCACAGTCAGTGAA-3′,
(57.7° C., 508 bp);
GAPDH
(SEQ ID NO: 6)
Forward: 5′-CTTTTAACTCTGGTAAAGTGG-3′,
GAPDH
(SEQ ID NO: 7)
Reverse: 5′-TTTTGGCTCCCCCCTGCAAAT-3′,
(56.3° C., 287 bp).

Western Blot Analysis

Cell lysates of the hMSC cultures derived from ALS and healthy individuals (ALS and non-ALS, respectively) were obtained with RIPA buffer (Sigma-Aldrich, MO, USA) in the presence of “Complete” protease inhibitor (Roche-Diagnostics, Basel, CH). Protein concentration was calculated by BCA protein assay kit (Pierce, Ill., USA). Protein samples of 15.5 μg were separated by electrophoresis through gradient 4-20% SDS-polyacrylamide gels (Bio-Rad laboratories, Hercules, CA, USA) and transferred to nitrocellulose membranes (GE Healthcare, NJ, USA), then blocked with 5% (w/v) non-fat milk in Trisbuffered saline/1% tween 20 (TBS/T) for 1 h. Primary antibodies were then added for overnight incubation at 4° C.; CyFIP2 (1:1000, Proteintech group Inc.), RbBP9 (1:1000, Proteintech group Inc.), β-actin as a loading control (1:2000, MP Biomedical, OH, USA), diluted in 5% Bovine serum albumin in TBS/T. Following three washes with TBS/T. Blots were probed with HRP-conjugated donkey anti-rabbit, donkey anti-mouse antibodies (Jackson Immunoresearch Laboratories, PA, USA) at 1:6000 in 5% Bovine serum albumin in TBS/T for 1 h at room temperature, following three washes with TBS/T. The detection was performed by ECL reaction (Thermo Fisher Scientific Inc, CA, USA). Protein levels were quantified by normalizing to the β-actin. Quantification of band intensities was performed by densitometry analysis using Quantity One® software (Bio-Rad laboratories).

Statistical Analysis

T-test with two sample equal variance after square root transformation was performed to establish statistical significance between the groups. A p value of 0.05 or less was judged to be significant, and results were expressed as mean ±standard deviation of average mean.

### Results

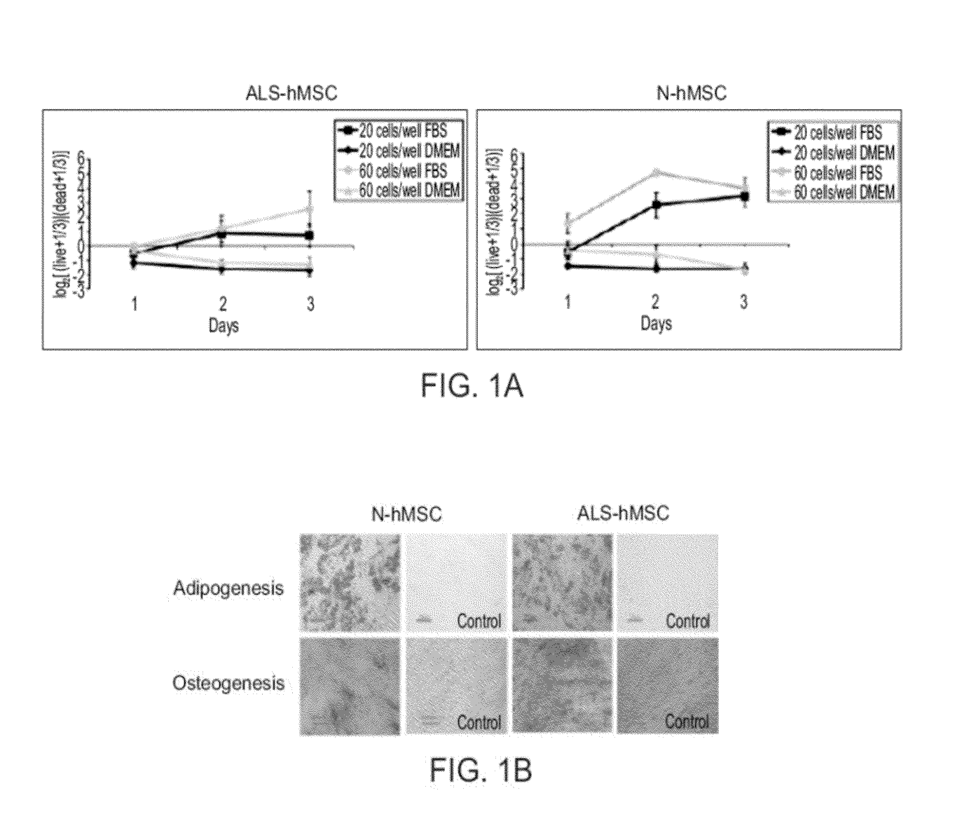

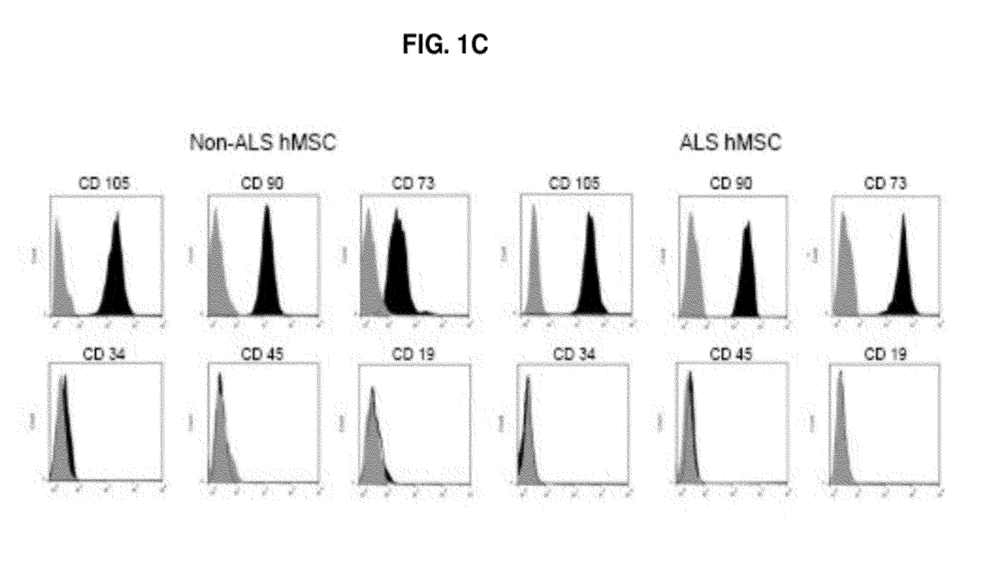

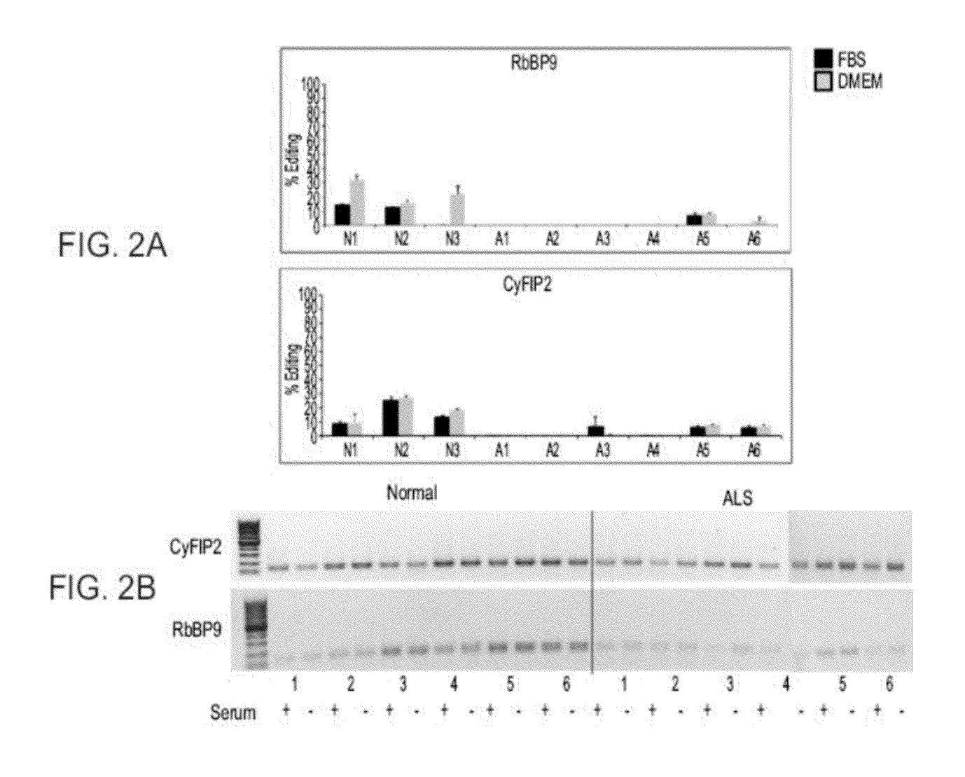

hMSC were isolated from bone marrow samples of 6 ALS patients and from similar number of healthy donors following their known property to adhere onto a plastic surface (Friedenstein et al., 1970, Cell Tissue Kinet. 3: 393-403). These isolated cells were routinely characterized (not shown) by the expression pattern of a number of known surface markers like CD29+, CD34−, CD44+, CD45−, and CD105+ using FACS flow cytometry as shown previously (Solmesky et al 2009, supra). These cells were grown until confluent and passages 3-7 were used for the experiments. Bone marrow derived hMSC are also characterized by their biological properties of prolonged survival under serum free conditions (Pochampally et al 2004 Blood 103:1647-1652; Solmesky et al., 2009, supra) and by their differentiation multipotency (Jiang et al., 2002, Nature 418:41-9). To this end, it was tested whether the ALS-hMSC maintain these basic biological features such as cell survival even at low densities under serum free conditions and their multi differentiation potential to produce osteoblasts and adipocytes compared to normal hMSC (FIGS. 1A-C). Overall, the biological features in ALS hMSC, surface markers expression (FIG. 1C), cell survival (FIG. 1A) and differentiation capacity (FIG. 1B), were found indistinguishable from non-ALS hMSC. FIG. 1A shows the results of a representative cell survival experiment with hMSC cultured at low density from 4 ALS and 2 unaffected donors performed for the period of three days. Cells were plated at a density of 20 or 60 cells per square millimeter in 24 well dishes in DMEM with the presence or absence of 10% serum. Cell viability in each well was determined at the end of the incubation period of 1, 2 and 3 days using vital fluorescent dye mix containing calcein-AM (stains living cells), propidium iodide (PI) (stains dead cells) and Hoechst 33342 (stains all nuclei) as described previously (Solmesky et al. 2009, supra). The results from these experiments indicate that ALS-hMSC survival is similar to unaffected controls under serum free conditions at high and at low cell densities. FIG. 1B shows the results of representative experiments of ALS-hMSC differentiation to either osteoblasts or adipocytes differentiation fates following established protocols (Pittenger et al., 1999, Science 284:143-147). No apparent difference in the differentiation potential was observed between normal hMSC and ALS-hMSC derived from each of the six ALS patients. Overall, these results indicate that ALS-hMSC in culture bare basic biological features of normal hMSC thus supporting the view that the biology of these stem cells is not affected by the disease.

To test the hypothesis that hMSC derived from ALS patients may serve as primary tools for ALS biomarker identification, ALS-hMSC were analyzed for the possibility that A to I conversion by RNA editing may be altered in specific target substrate genes of the ADAR-mediated RNA editing enzymes (Levanon, et al., 2005, Nucleic Acids Res. 33: 1162-68). RNA editing levels in ALS and non-ALS hMSC of 10 target genes were analyzed that are known to undergo A-I RNA editing (58, 62) which are: FANCC, BLCAP, BRCA1, CyFIP2, RbBP9, MDM4, FLNA, GluR-2 (the Q/R site), CARD11 and ALS2CR8 (Table 1 below).

TABLE I

| | Forward primer for PCR | Reverse primer for PCR | Extension |
|---|---|---|---|
| | amplification of the edited | amplification of the edited | primer/ |
| Gene | region/(SEQ ID NO:) | region(SEQ ID NO:) | (SEQ ID NO:) |
| BLCAP | 5′ACGTTGGATG- | 5′ACGTTGGATG- | 5′GCAGCCACTGGA |
| | ATTAGGTCGGTTCCTGCAGC | AGGAGGACGGGCA | GGCAA/10 |
| | ACGTTGGATGATTAGGTCG/8 | GCAACGTTG | |
| | | GATGAGGAGGAC/9 | |
| CYFIP2 | 5′ACGTTGGATG- | 5′ACGTTGGATG- | 5′CTCTTCATAGTG |
| | TTTCGGCGACATGCAGATAG/11 | ACGTCCACTTGGACTTGTTC/12 | AGCACTGGTCT/13 |
| FLNA | 5′ACGTTGGATG- | 5′ACGTTGGATG- | 5′TCACTGTTTCTA |
| | TTCGTGGTGCCTGTGGCTT/14 | TGGGCGGTTTCTCTCGGTG/15 | GCCTTC/16 |
| RBBP9 | 5′ACGTTGGATG- | 5′ACGTTGGATG- | 5′GGTGGCTCATGC |
| | CCTTTTTTTGGCTGGGTGTG | GCCTCAAGCAATACAACCAC | CTGTA/19 |
| | ACGTTG GAT GCCTTTTTT/17 | ACG TTG GAT GGCCTC | |
| | | AA/18 | |
| CARD11 | 5′ACGTTGGATG- | 5′ACGTTGGATG- | 5′CAGCCTGGTGAC |
| | AGATCACGCCACTGCACTC/20 | CCAGGTCAAGCTTGCCTTTT/21 | AGAGC/22 |
| MDM4 | 5′ACGTTGGATG- | 5′ACGTTGGATG- | 5′AGTGGCTCACGC |
| | AAAAAAATCGTGGACCGGGC/23 | ACCTAGGTGATCTCCCAAAG/24 | CTGTA/25 |
| BRCA1 | 5′ACGTTGGATG- | 5′ACGTTGGATG- | 5′GTCGCCCAGGTT |
| | TATTTTGAGATGGAGTCCAG | GAAGTGCACGTTGCAGTGA | GGAGT/28 |
| | C/26 | G/27 | |
| FANCC | 5′ACGTTGGATG- | 5′ACGTTGGATG- | 5′AGAGATGGGGTT |
| | GGACTGGAGGAAGCACTAAT/29 | TGAGCCTCAGAGGTTGAGAC/30 | TCACC/31 |

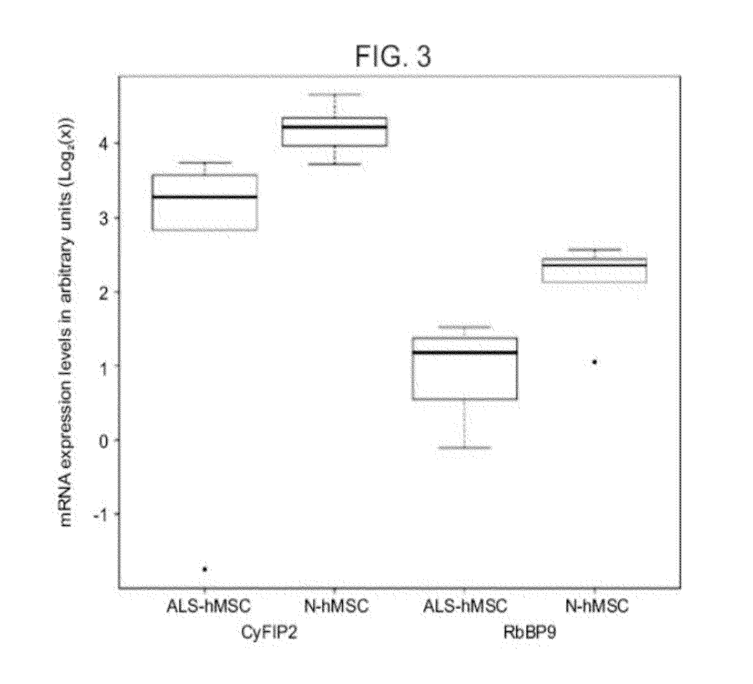

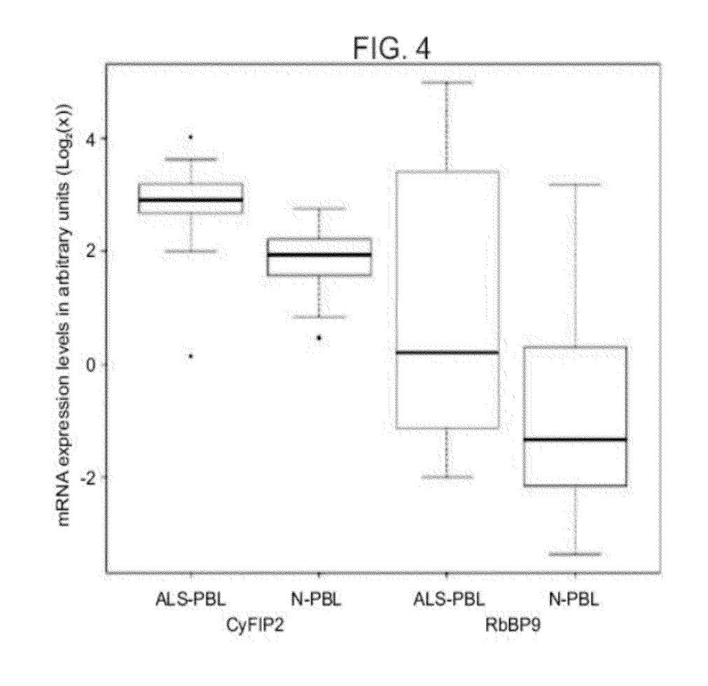

These results lead to the investigation of the mRNA expression levels of both genes in ALS-hMSC and in control hMSC cultures using quantitative real time PCR analysis. In these experiments significant quantitative differences were found of expression for both genes CyFIP2 and RbBP9 between ALS and normal hMSC as shown in FIG. 3. Altogether, the ALS specific RNA editing pattern found consistently in all ALS individuals for CyFIP2 and RbBP9 and the differences found at the transcriptional level of these genes strongly support our view that ALS-hMSC are useful model tool to identify possible ALS biomarkers. To strengthen the possibility that these genes could become future diagnostic ALS biomarkers, several experiments were performed on peripheral blood samples of ALS and healthy individuals. First RT-PCR analysis was done to verify the expression of these genes in PBL. For this purpose RNA extracts were made from isolated PBL from blood samples of 9 ALS individuals and from blood samples of 8 healthy donors. RT-PCR analysis from these experiments indicate that the two genes CyFIP2 and RbBP9 are expressed in WBC of normal and ALS individuals (data not shown). Then an RNA editing analysis was done on these RNA samples and found that both genes are not RNA edited in blood while the control gene BLCAP showed similar RNA editing levels in all blood samples (data not shown). Having determined that CyFIP2 and RbBP9 are not post transcriptionally modified by A-I RNA editing in blood, nevertheless, the possibility that these genes are differentially expressed in ALS blood samples as shown above in ALS-hMSC (see FIG. 3) was investigated. Thus quantitative real time PCR experiments were performed to determine the mRNA expression levels of CyFIP2 and RbBP9 in PBL isolates from blood samples of a total of 17 ALS and 19 healthy individuals. FIG. 4 shows the results from these experiments a significant difference was detected in the mRNA expression levels of both genes in ALS-PBL compared to the control PBL population. These results found in ALS-PBL may support that the RNA deficiently edited genes CyFIP2, RbBP9 identified in ALS-hMSC, could serve as diagnostic biomarker tools for detection of ALS in blood samples. Following these experiments inventors examined the protein expression levels of CyFIP2, RbBP9 by Western blot analysis of ALS and non-ALS hMSC protein extracts. FIGS. 5A-B show that the protein levels of CyFIP2 and RbBP9 were significantly lower in all ALS hMSC tested compared to non-ALS hMSC.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. A method of diagnosing amyotrophic lateral sclerosis (ALS) in a subject in need thereof, the method comprising determining in cells of the subject at least one alteration in a sequence or expression level of Cytoplasmic FMR Interacting Protein (CyFIP2; Accession AF160973) and/or Retinoblastoma Binding Protein 9 (RbBP9; Accession AF039564), wherein an altered sequence or expression of said sequence or expression level of said CyFIP2; Accession AF160973) or Retinoblastoma Binding Protein 9 (RbBP9; Accession AF039564) as compared to a control reference sample from a non-ALS subject, is indicative of ALS.

2. The method of claim 1, wherein said cells comprise mesenchymal stem cells.

3. The method of claim 1, wherein said cells comprise peripheral white blood cells.

4. The method of claim 2, wherein said alteration in a sequence or expression level comprises an alteration in RNA editing.

5. The method of claim 1, wherein said determining said alteration is effected by an assay selected from the group consisting of PCR, RNA editing assay and PAGE analysis.

6. The method of claim 5, wherein said RNA editing assay comprises direct sequencing.

7. The method of claim 5, wherein said RNA editing assay assays an A>I editing.

8. The method of claim 3, wherein said alteration in expression level comprises increased expression level.

9. The method of claim 2, wherein said alteration in expression level comprises decreased expression level.

10. The method of claim 1, wherein said determining said expression level is effected at the mRNA level.

11. The method of claim 1, further comprising substantiating said diagnosis using a diagnosis method selected from the group consisting of electromyography, nerve conduction velocity magnetic resonance imaging (MRI) and bio-molecular analysis.

12. The method of claim 1, further comprising planning a treatment regimen for said ALS following said step (a).

13. The method of claim 1 further comprising (b) informing the subject of ALS diagnosis.

14. A kit for diagnosing ALS comprising means container including:
at least a pair of oligonucleotide primers capable of specifically binding to any one of CyFIP2 and RbBP9 mRNA sequences or the complement thereof;
antibodies capable of specifically binding to any one of CyFIP2 and RbBP9.

15. A method of screening for a medicament for ALS, the method comprising:
(a) treating cells of an ALS patient with a putative ALS medicament, so as to obtain treated cells; and
(b) determining in said treated cells at least one alteration in a sequence or expression level of Cytoplasmic FMR Interacting Protein (CyFIP2; Accession AF160973) and/or Retinoblastoma Binding Protein 9 (RbBP9; Accession AF039564), wherein an altered sequence or expression of said sequence or expression level of said CyFIP2; Accession AF160973) or Retinoblastoma Binding Protein 9 (RbBP9; Accession AF039564) as compared to a control reference of non-treated cells of said ALS patient, wherein said alteration is indicative of a medicament for ALS.

16. The method of claim 15, wherein said cells comprise mesenchymal stem cells.

17. The method of claim 16, wherein said alteration comprises up-regulation of said expression and RNA editing.

18. The method of claim 15, wherein said cells comprise peripheral blood cells.

19. The method of claim 18, wherein said alteration comprises down-regulation of said expression and up-regulation of RNA editing.

20. A method of treating ALS in a subject in need thereof, the method comprising:
(a) obtaining cells of the subject;
(b) treating said cells with an ALS medicament, so as to obtain treated cells;
(c) determining in said treated cells at least one alteration in a sequence or expression level of Cytoplasmic FMR Interacting Protein (CyFIP2; Accession AF160973) and/or Retinoblastoma Binding Protein 9 (RbBP9; Accession AF039564), wherein an altered sequence or expression of said sequence or expression level of said CyFIP2; Accession AF160973) or Retinoblastoma Binding Protein 9 (RbBP9; Accession AF039564) as compared to a control reference of non-treated cells of said ALS patient, wherein said alteration is indicative of an efficacious medicament for ALS; and
(d) treating the subject with said efficacious medicament.

21. The method of claim 1, wherein said determining said expression level is effected at the protein level.


SEQUENCE LISTING (36 sequences)

SEQ ID NO: 1 - DNA, 23 bp, Artificial sequence
  Single strand DNA oligonucleotide
ctttcccatatacaagttctcca

SEQ ID NO: 2 - DNA, 20 bp, Artificial sequence
  Single strand DNA oligonucleotide
gctggttcatcccacagaca

SEQ ID NO: 3 - DNA, 20 bp, Artificial sequence
  Single strand DNA oligonucleotide
aggacaatgggtccatccag

SEQ ID NO: 4 - DNA, 20 bp, Artificial sequence
  Single strand DNA oligonucleotide
gctgcactcatggcttctcc

SEQ ID NO: 5 - DNA, 21 bp, Artificial sequence
  Single strand DNA oligonucleotide
aaagtggccacagtcagtgaa

SEQ ID NO: 6 - DNA, 21 bp, Artificial sequence
  Single strand DNA oligonucleotide
cttttaactctggtaaagtgg

SEQ ID NO: 7 - DNA, 21 bp, Artificial sequence
  Single strand DNA oligonucleotide
ttttggctcccccctgcaaat

SEQ ID NO: 8 - DNA, 49 bp, Artificial sequence
  Single strand DNA oligonucleotide
acgttggatgattaggtcggttcctgcagcacgttggatgattaggtcg

SEQ ID NO: 9 - DNA, 44 bp, Artificial sequence
  Single strand DNA oligonucleotide
acgttggatgaggaggacgggcagcaacgttggatgaggaggac

SEQ ID NO: 10 - DNA, 17 bp, Artificial sequence
  Single strand DNA oligonucleotide
gcagccactggaggcaa

SEQ ID NO: 11 - DNA, 30 bp, Artificial sequence
  Single strand DNA oligonucleotide
acgttggatgtttcggcgacatgcagatag

SEQ ID NO: 12 - DNA, 30 bp, Artificial sequence
  Single strand DNA oligonucleotide
acgttggatgacgtccacttggacttgttc

SEQ ID NO: 13 - DNA, 23 bp, Artificial sequence
  Single strand DNA oligonucleotide
ctcttcatagtgagcactggtct

SEQ ID NO: 14 - DNA, 29 bp, Artificial sequence
  Single strand DNA oligonucleotide
acgttggatgttcgtggtgcctgtggctt

SEQ ID NO: 15 - DNA, 29 bp, Artificial sequence
  Single strand DNA oligonucleotide
acgttggatgtgggcggtttctctcggtg

SEQ ID NO: 16 - DNA, 18 bp, Artificial sequence
  Single strand DNA oligonucleotide
tcactgtttctagccttc

SEQ ID NO: 17 - DNA, 48 bp, Artificial sequence
  Single strand DNA oligonucleotide
acgttggatgcctttttttggctgggtgtgacgttggatgcctttttt

SEQ ID NO: 18 - DNA, 47 bp, Artificial sequence
  Single strand DNA oligonucleotide
acgttggatggcctcaagcaatacaaccacacgttggatggcctcaa

SEQ ID NO: 19 - DNA, 17 bp, Artificial sequence
  Single strand DNA oligonucleotide
ggtggctcatgcctgta

SEQ ID NO: 20 - DNA, 29 bp, Artificial sequence
  Single strand DNA oligonucleotide
acgttggatgagatcacgccactgcactc

SEQ ID NO: 21 - DNA, 30 bp, Artificial sequence
  Single strand DNA oligonucleotide
acgttggatgccaggtcaagcttgcctttt

SEQ ID NO: 22 - DNA, 17 bp, Artificial sequence
  Single strand DNA oligonucleotide
cagcctggtgacagagc

SEQ ID NO: 23 - DNA, 30 bp, Artificial sequence
  Single strand DNA oligonucleotide
acgttggatgaaaaaaatcgtggaccgggc

SEQ ID NO: 24 - DNA, 30 bp, Artificial sequence
  Single strand DNA oligonucleotide
acgttggatgacctaggtgatctcccaaag

SEQ ID NO: 25 - DNA, 17 bp, Artificial sequence
  Single strand DNA oligonucleotide
agtggctcacgcctgta

SEQ ID NO: 26 - DNA, 31 bp, Artificial sequence
  Single strand DNA oligonucleotide
acgttggatgtattttgagatggagtccagc

SEQ ID NO: 27 - DNA, 30 bp, Artificial sequence
  Single strand DNA oligonucleotide
acgttggatggaagtgcacgttgcagtgag

SEQ ID NO: 28 - DNA, 17 bp, Artificial sequence
  Single strand DNA oligonucleotide
gtcgcccaggttggagt

SEQ ID NO: 29 - DNA, 30 bp, Artificial sequence
  Single strand DNA oligonucleotide
acgttggatgggactggaggaagcactaat

SEQ ID NO: 30 - DNA, 30 bp, Artificial sequence
  Single strand DNA oligonucleotide
acgttggatgtgagcctcagaggttgagac

SEQ ID NO: 31 - DNA, 17 bp, Artificial sequence
  Single strand DNA oligonucleotide
agagatggggtttcacc

SEQ ID NO: 32 - DNA, 20 bp, Artificial sequence
  Single strand DNA oligonucleotide
acccttactgagctcccttc

SEQ ID NO: 33 - DNA, 6791 bp, Homo sapiens
ggacccgccgcagagccaggtaagcggccctcgcaggcctcgggccgggcacggggactaggatgccgccggggacggggatgaggccgtgaggatgcgcgaagggccgccccctctcccggcccgcggggggcgctcggcgctgtgcccgggctagcccgaggcccggcctcaggccccacgcggcccctttccccctcggacccaccgtgcgtcccgcggcacggaccctctgcccgggaggcgcgggcacatcgcggagctccggcgcggcggcgggggagccgcagcagcaggtgcgcggcctgggccggagccgccagcccgggaggaggcggtgctaatctcagggaccggagacacctgcagcggccgcgagcccggcagcggcgacatcctcgagtccagtgcagaatacagaaactgcagccatgaccacgcacgtcaccctggaagatgccctgtccaacgtggacctgcttgaagagcttcccctccccgaccagcagccatgcatcgagcctccaccttcctccatcatgtaccaggctaactttgacacaaactttgaggacaggaatgcatttgtcacgggcattgcaaggtacattgagcaggctacagtccactccagcatgaatgagatgctggaggaaggacatgagtatgcggtcatgctgtacacctggcgcagctgttcccgggccattccccaggtgaaatgcaacgagcagcccaaccgagtagagatctatgagaagacagtagaggtgctggagccggaggtcaccaagctcatgaagttcatgtattttcagcgcaaggccatcgagcggttctgcagcgaggtgaagcggctgtgccatgccgagcgcaggaaggactttgtctctgaggcctacctcctgacccttggcaagttcatcaacatgtttgctgtcctggatgagctaaagaacatgaagtgcagcgtcaagaatgaccactctgcctacaagagggcagcacagttcctgcggaagatggcagatccccagtctatccaggagtcgcagaacctttccatgttcctggccaaccacaacaggatcacccagtgtctccaccagcaacttgaagtgatcccaggctatgaggagctgctggctgacattgtcaacatctgtgtggattactacgagaacaagatgtacctgactcccagtgagaaacatatgctcctcaaggtgatgggctttggcctctacctaatggatggaaatgtcagtaacatttacaaactggatgccaagaagagaattaatcttagcaaaattgataaattctttaagcagctgcaggtggtgccccttttcggcgacatgcagatagagctggccagatacattaagaccagtgctcactatgaagagaacaagtccaagtggacgtgcacccagagcagcatcagcccccagtacaatatctgcgagcagatggttcagatccgggatgaccacatccgcttcatctccgagctcgctcgctacagcaacagtgaggtggtgacgggctcagggctggacagccagaagtcagacgaggagtatcgcgagctcttcgacctagccctgcggggtctgcagcttctatccaagtggagcgcccacgtcatggaggtgtactcttggaagctggttcatcccacagacaagttctgcaacaaggactgtcctggcaccgcggaggaatatgagagagccacacgctacaattacaccagtgaggaaaaatttgccttcgttgaggtgatcgccatgatcaaaggcctgcaggtgctcatgggcaggatggagagcgtcttcaaccaggccatcaggaacaccatctacgcggcattgcaggacttcgcccaggtgacgctgcgtgagcccctgcggcaggcggtacggaagaagaagaatgtcctcatcagcgtcctacaggcaattcgaaagaccatctgtgactgggagggagggcgagagccccctaatgacccatgcttgagaggggagaaggaccccaaaggtggatttgatatcaaggtgccccggcgtgctgtggggccatccagcacacagctgtacatggtgcggaccatgcttgaatcactcattgcagacaaaagcggctccaagaagaccctgaggagcagcctggatggacccattgtcctcgccatagaggactttcacaaacagtccttcttcttcacacatctgctcaacatcagtgaagccctgcagcagtgttgtgacctctcccagctctggttccgagaattcttcctggagttaaccatgggccgacgaatccagttccccatcgagatgtccatgccctggattctaacggaccatatcctggaaaccaaagaaccttccatgatggagtatgtcctctaccctctggatctgtacaacgacagcgcctactatgctctgaccaagtttaaaaagcagttcctgtacgatgagatagaagctgaggtgaacctgtgttttgatcagtttgtctacaagctggcagaccagatctttgcttactacaaagccatggctggcagtgtcctgttggataaacgttttcgagctgagtgtaagaattatggcgtcatcattccgtatccaccgtccaatcgctatgaaacactgctgaagcagagacacgtccagctgttgggtagatcaattgacttgaacagactcattacccagcgcatctctgccgccatgtataaatccttggaccaagctatcagccgctttgagagtgaggacctgacctccattgtggagctggagtggctgctggagattaaccggctcacgcatcggctgctctgtaagcatatgacgctggacagcttcgatgccatgttccgagaggccaatcacaatgtgtccgccccctatggccgtatcaccctgcatgtcttctgggaactgaactttgactttctccccaactactgctacaatgggtccactaaccgttttgtgcggactgccattcctttcacccaagaaccacaacgagacaaacctgccaacgtccagccttattacctctatggatccaagcctctcaacattgcctacagccacatctacagctcctacaggaatttcgtggggccacctcatttcaagactatctgcagactcctgggttatcagggcatcgctgtggtcatggaggaactgctaaagattgtgaagagcttgctccaaggaaccattctccagtatgtgaaaacactgatagaggtgatgcccaagatatgccgcttgccccgacatgagtatggctccccagggatcctggagttcttccaccaccagctgaaggacatcattgagtacgcagagctcaaaacagacgtgttccagagcctgagggaagtgggcaatgccatcctcttctgcctcctcatagagcaagctctgtctcaggaggaggtctgcgatttgctccatgccgcacccttccaaaacatcttgcctagagtctacatcaaagagggggagcgcctggaggtccggatgaaacgtctggaagccaagtatgccccgctccacctggtccctctgatcgagcggctggggacccctcagcaaatcgccattgctcgcgagggtgacctcctgaccaaggagcggctgtgctgtggcctgtccatgttcgaggtcatcctgacccgcattcggagctacctgcaggaccccatctggcggggcccaccgcccaccaatggcgtcatgcacgtcgatgagtgtgtggagttccaccggctgtggagcgccatgcagttcgtgtactgcatccctgtgggaaccaacgagttcacagctgagcagtgtttcggcgatggcttgaactgggctggttgctccatcattgtcctgctgggccagcagcgtcgctttgacctgttcgacttctgttaccacctgctaaaagtgcagaggcaggacgggaaggatgaaatcattaagaatgtgcccctgaagaagatggccgaccggatcaggaagtatcagatcttgaacaatgaggtttttgccatcctgaacaaatacatgaagtccgtggagacagacagttccactgtggagcatgtgcgctgcttccagccacccatccaccagtccttggccaccacttgctaagcagaagatcctgcagacccttatctggaggaggaagagaagcaggagagagaaagccacagccagcctgccataggatccaactggacaacgtgtgggatggacctggaaacaagcacctccccaaacacatcaccactccctagggcggggcctgtgcatgctctcccatgacatctccatgctggtttctccatagcataaatgaaaaaaaaaaaaaaaaagtaaacagggcagtgtgtgctttttcttttctcccccctcaactatattaagaactcctagtttcaccctttctccatcccatcatcccacctatctgtggttgcttcccaagacctcctcccaagatagacatctcctacccagtgcccttgtgtgaccccaggactcaagtctcagactgtgaacagatgtggccatgcccagagacgccagcctggccagaagggcatgcctcagcttactacttcatctctcctggttccctccctgcagtgccccgggtgtcatcttctcccactctgggtaccagggattctaccacataggcttcccaaagccccattctaactcccctctctcagggaagccctagagagaggtccaaaaagcattcacagctgtatcacactctatgcaggtggggtaggagactgatcaggcctgctgtggggaagcagtatgtatgaacacagccagaaatgtcatagtccaaacaggatgctttcaggccatctcagctgcttgatggtgagatggttcccttattccttcaggaaaggcttagcattgggccacataggggaagcagctttgaacaaatcagtcatagcactgcctatagcattagccagtgaccaaattagggacaacgtcttggcacagaattgcttatcaaggaacatttccacaagaaagaaaatattaaggggttatttccacagaagcccaaaacgtcttggaaacacagaggtgaggaggaggaatagtaattgtcaatgagcttttaataccaagatacaccccctgcccccaaagaagagtcctcttttagggaatcagaaccttcattgtcctagaagctgaaagattcttggaacattttagcttttactctcaacttgctgttctctttacattccttaagttagactttcgggtgtggcttctctcccaggggtaacatttacttccattttctagactgaaccaaaagtcttctgcagaatctcccaccgagtgtggtaagaaggaaggacaaaaggctttaggatataaatttcatgttacagagcatgtcattgtcaaaggaaatctgtggccctgagattttaagaacataaaatgtgacatttgatatttctccagcccagggaagtaagatggttagcaatggttgccttaatcaaatggtcccatttttaaccccaaaggaagtgcccacagcaagaggtttgtgtgatgcacttatgtcctccggtgaggaaagggggccacatatgaaaggccccttaggtcagatcctgagagtagcacatttgagtgcagattcctgggccccacctcaaacctactaattctgaatctctgggaatagggccaggaaatctgccctttctacaaactacccaagttgttctgttgcacatcaatgtttgggaaccactgctgtaagggaatcattctggtcaccttgagctttgagctaccactaagccatgaaagaaaatacatcatacagggaagagagaagggaggaggttccaagtagtaactggcagatcctcctgtctggaggtaccaccttctattctggtttctgacttttccttcttgatgaccatagatgtgttccagaggcaaaagagacacattatcccagatggcagaacatgctttcaaaacatataaaatgtcaaagttccagatccttctacatctttagtcctgtctgaggatggtagctggctctctgtagctgatagatggctagagttccatccaaatccttgaccacgacttcatggagatttgaataatctatttgatgagatttctatttcaataacccacctctctcaccccacattcatatccctaaatttgaccctctgggccgagtcacattaccttcaggagacttgatcccagtagactgaggtcttccctttcagcagaaagatttcatttccctggcttgccagtggcactgatttccgaacacccaatgagtttaatattctttcctccttggcattactgccccagcctctttttattttttttgtgtgtgtctaataaccaggaaaaaaataaagcttaggttttaaaaagttttaaaaataatctgtttcagaaactgtcaaatgtaccatatttgtattaagagttgttgggaatttttgtacaatgaatttacatttatttatggtgacatatttacgcttgtgatcaaataatgatgttaaattcttaaatcatatttgctatgcagctgaagatgatattttgatttgtattttgggggtacctgtgttgagttgataaacatttccatcttcattaaaactgcttccaaactagtaaaaccagcaa

SEQ ID NO: 34 - protein, 1253 aa, Homo sapiens
MTTHVTLEDALSNVDLLEELPLPDQQPCIEPPPSSIMYQANFDTNFEDRNAFVTGIARYIEQATVHSSMNEMLEEGHEYAVMLYTWRSCSRAIPQVKCNEQPNRVEIYEKTVEVLEPEVTKLMKFMYFQRKAIERFCSEVKRLCHAERRKDFVSEAYLLTLGKFINMFAVLDELKNMKCSVKNDHSAYKRAAQFLRKMADPQSIQESQNLSMFLANHNRITQCLHQQLEVIPGYEELLADIVNICVDYYENKMYLTPSEKHMLLKVMGFGLYLMDGNVSNIYKLDAKKRINLSKIDKFFKQLQVVPLFGDMQIELARYIKTSAHYEENKSKWTCTQSSISPQYNICEQMVQIRDDHIRFISELARYSNSEVVTGSGLDSQKSDEEYRELFDLALRGLQLLSKWSAHVMEVYSWKLVHPTDKFCNKDCPGTAEEYERATRYNYTSEEKFAFVEVIAMIKGLQVLMGRMESVFNQAIRNTIYAALQDFAQVTLREPLRQAVRKKKNVLISVLQAIRKTICDWEGGREPPNDPCLRGEKDPKGGFDIKVPRRAVGPSSTQLYMVRTMLESLIADKSGSKKTLRSSLDGPIVLAIEDFHKQSFFFTHLLNISEALQQCCDLSQLWFREFFLELTMGRRIQFPIEMSMPWILTDHILETKEPSMMEYVLYPLDLYNDSAYYALTKFKKQFLYDEIEAEVNLCFDQFVYKLADQIFAYYKAMAGSVLLDKRFRAECKNYGVIIPYPPSNRYETLLKQRHVQLLGRSIDLNRLITQRISAAMYKSLDQAISRFESEDLTSIVELEWLLEINRLTHRLLCKHMTLDSFDAMFREANHNVSAPYGRITLHVFWELNFDFLPNYCYNGSTNRFVRTAIPFTQEPQRDKPANVQPYYLYGSKPLNIAYSHIYSSYRNFVGPPHFKTICRLLGYQGIAVVMEELLKIVKSLLQGTILQYVKTLIEVMPKICRLPRHEYGSPGILEFFHHQLKDIIEYAELKTDVFQSLREVGNAILFCLLIEQALSQEEVCDLLHAAPFQNILPRVYIKEGERLEVRMKRLEAKYAPLHLVPLIERLGTPQQIAIAREGDLLTKERLCCGLSMFEVILTRIRSYLQDPIWRGPPPTNGVMHVDECVEFHRLWSAMQFVYCIPVGTNEFTAEQCFGDGLNWAGCSIIVLLGQQRRFDLFDFCYHLLKVQRQDGKDEIIKNVPLKKMADRIRKYQILNNEVFAILNKYMKSVETDSSTVEHVRCFQPPIHQSLATTC

SEQ ID NO: 35 - DNA, 3871 bp, Homo sapiens
cgcaggctccctgcgggagcaggctgggctcagctccgctggacccgcgctggggaactctggcctcgctgcactcatggcttctcctagcaaggcagtgattgttcccgggaacggaggcggggatgtgaccacccacggctggtatggctgggtgaaaaaggagctggagaagatacctggtttccagtgtttggctaaaaacatgcccgacccaattacagcacgagagagcatctggctgcccttcatggagacagagctgcactgtgatgagaagactatcatcattggccacagttctggggccatcgcggccatgaggtatgcagaaacacatcgagtatatgctattgtattagtgtctgcgtacacatcagacttgggggatgaaaatgagcgtgcaagtggatacttcacccgcccctggcagtgggagaagatcaaggccaactgcccttacattgtgcagtttggctctactgacgacccgttccttccctggaaggaacaacaagaagtggccgataggttggaaaccaaattgcacaaattcactgactgtggccactttcagaacacagagtttcatgaactgattactgttgtaaagtctttgctgaaagtaccagcatagactgtatgatttctgctattttgcatcccaatataggagtagaataatatctactatcagctgattactagacacatagaacatccagttaagttccgtaagtgcctgaaaaacaaacacaagtttcaacattcaatctaagttacaaatagcacttccattttccatagactctaaaactccccaaattgctatgaactaccacagtattttcctcatttgataagaaacataaggacagcttaactacccttctctatccgaagtgagtcagaaacagaacccaggtttcttgattcccactcctgagtaaaattaaataggttttatgtatgttgcaggtattgcacacttttcctctttgaaatcaaacttttctatttcactggggaaagaaaacatgactgaaagtccagagtgttagcttagagctcaaaatactagctgctgtttcaagctggccccagacattaaactcagactagggccttcacctctgacacttgcccctaagtccttcagacatgttgagagagactgagacaggagtcttccggagtcaggcaggaaggagaagcacagcaatatacattcataatatacgtacaactgactagatttgtttgagacaagagtggtgggatcagagccatgtggagagctcatggcctgtctaaattgttgccatgtggaaatggggcagagtgttgccaattttctgattttttgtgtggaaaaaaatctccaaattgtagtttttaaatttggggaatgcaaatatatatatatatacacacacacacacacacatacacatacaatccatatacatatggagtatgtaagttggcaataacagtatacatcaaaaaatttacaaacactgcaagacaaataaaatccaagccatccatttgtgaccacagtcataagcattcacttagccaggggcttttagataattgtctgtgaagtctgagaaaagactaagataaccaagatgaacataatgtcaaaggacgaaatcatggggacaatttggattttgttccaaaagacaagtgtttttaaaaagaacctttccatacttcgtgttctgttctatagagttaaaccttttaacttccatggccctttagatctctttcaagcacattccattttcttctgatcatacttcctcagaaaacctttttttggctgggtgtggtggctcatgcctgtaatcccagcactgtgggaggctgaggtggttgtattgcttgaggccaggtgtttgaggccagccagggcaacacagcaagaccccatctctacaaaatattaaaacattagctaggcatggtggtgcacacctgtagtcctagcttcttgggaggctgaggcaggattgcttgagcccaggagtttgagccttcagtgagctatgatcgcgccactgtactccatcctgggtgatggagcgaagaacctgtctcttaaaaaaaaaaatttttttttaattaaaaaaacccaccctttttcatttcccaacattttttggctattatgatcgctatgtgttccttcaaataatttccagtgaatccacacttgatgttgcaaagcctaagtttgctgcagaaatacaatttcctacacttcatacctaaaggagattcagagctatcgctcaaaaccaaaagtcttggtttttaaatgtgcttagacaaaaaaaatcaggaaaggtaccctaatcgttcaacagcaatcatctttggggaatgggcattttggtaggtaaagtgtggtggacgtccttattattatttttgagacagtgtcttgctctgtctcccaggctggaggtcagtggcacaaatctcagctcactctaacctgcacctcccgggttcaagtgattctcatctcagcctcccaagtagtgtgaactaaaggcatgtgccaccacgcctggctaatttttgtatttttagtagagacagggtttcaccatgttggccaggctggtctcgaactcctggcctcaagtgatccacccacctcagccgcccaaagtgttgggattacaggcatgagccaccatgcccggccccttattttactttatcctcctgattatttgaatttgttacactaagcatgtattttataatcaaaaataattaagatttaaaacaatgtatttgactactaaaaagcaaattatataaatcagttgaataataatgttgttaaagaggctgtttagcactaatttgaccaaatctatagtcaaagtttgattctacttgaattttttggaaatttgctgttgtattacttattctttgccttgttctctgtgaagtcctttcctaaaaaagaaacttatacaatttagttcacaaaactttgttgacctctttctctgtagaaatgaaaactaaatatatataattgagaaataattctgatactcttcattcctataaaagtaattaagatagacctataggattgttgaactttgctgccaaatatagtaaccagtaaccacagctagctgtaaaattgaaaattcagttcagtcaaactagccatatttcaagtgctcaacagccactggtggctattggttaccttactgaccagccaatataggctatttccgtcattatagaaagctctattggacaacattggtctaggacatatattttgtattttcccctcacccaccatccaataatgagtaatagcacaaagtaaaagtgacttaatttgaaattacttctgtttggctagtttgcttagctgatcaccaccaacatcataggtctataaatcacccaactaagatcaggggtgtttcatgggaaaatctacacaaactattcattaaaaaccatagagccatggactgtcatttctgatttctgtttgattctttgtggtcatcattgaatatacaggggcctctatacactctggggagtatctattctcaggtgagcaaaataccgaatactgaatggaaatcaactgtaagtattaggacttcatatgcaacttgaatttttggtgctgtccggagtctagagccccacaatctgttttggttacagtttatccctgtaggataaatgatccatttaactattcatcagaggtgctgtaattttaaattgtctcttgtctccttcagttaattttcagaattaaaaacataccatgggaaaaaaaaaaaaaaaa

SEQ ID NO: 36 - protein, 186 aa, Homo sapiens
MASPSKAVIVPGNGGGDVTTHGWYGWVKKELEKIPGFQCLAKNMPDPITARESIWLPFMETELHCDEKTIIIGHSSGAIAAMRYAETHRVYAIVLVSAYTSDLGDENERASGYFTRPWQWEKIKANCPYIVQFGSTDDPFLPWKEQQEVADRLETKLHKFTDCGHFQNTEFHELITVVKSLLKVPA